(19) United States
(12) Patent Application Publication
(10) Pub. No.: US 2002/0143075 A1
(43) Pub. Date: Oct. 3, 2002

(54) Low-profile moving bed reactor

(76) Inventors: Kenneth L. Agee, Bixby, OK (US); Larry J. Weick, Tulsa, OK (US)

Correspondence Address: John E. Vick, Jr., Dority & Manning, Attorneys at Law, P.A., P.O. Box 1449, Greenville, SC, 29602, US

(21) Appl. No.: 09/818,277

(22) Filed: Mar. 27, 2001

(51) Int. Cl. C07C 27/06; B32B 27/04

(52) U.S. Cl. 518/710; 422/140

(57) ABSTRACT

The invention is directed to reactor systems, apparatus, and processes which are useful for conducting chemical reactions that may be effected in a three phase slurry system. One particular application of the invention converts synthesis gas (syngas) into hydrocarbons. Syngas is comprised of carbon monoxide and hydrogen. In general, a low profile bed reactor is capable of conducting an exothermic catalytic conversion. The reactor may also include a catalyst contained in a moving fluid system which ascends in the reactor in one or more stages. A heat exchanger optionally may be used to remove heat, and water may be removed from the reaction as it proceeds from one stage to another. The reactor is designed in a relatively low profile horizontal design, and is usually more efficient and inexpensive to operate (or build) than taller vertically oriented reactors of the same type.

### FIELD OF THE INVENTION

The invention relates to reactors, processes and systems useful in conducting chemical reactions in a slurry phase. In particular, the invention relates to low-profile moving bed reactors.

### BACKGROUND OF THE INVENTION

Numerous chemical reactions may be performed in a slurry phase. Slurry bubble column reactors comprise vertical cylindrical vessels that are used in such slurry phase reactions. For example, U.S. Pat. No. 5,348,982 is directed to one example of a slurry bubble column reactor that is used in the context of a Fischer-Tropsch reaction.

The synthetic production of hydrocarbons by the catalytic reaction of synthesis gas is well known and is generally referred to as the Fischer-Tropsch reaction (“F-T” reaction). The Fischer-Tropsch reaction was developed in the early part of the twentieth century in Germany. It was practiced commercially in Germany during World War II, and later was practiced in South Africa.

Reactors and process vessels contain chemical reactants during a chemical conversion process. Reactors may be catalyzed or non-catalyzed. Because a reaction may be exothermic or endothermic, a heat exchanger frequently is provided in the reactor design to provide heat or to absorb heat.

The FT reaction refers to the synthetic production of hydrocarbons by the catalytic reaction of syngas, which is primarily carbon monoxide and hydrogen. Once the syngas is produced, it is treated and delivered to a synthesis reactor, where heavier hydrocarbons are formed. The synthesis reaction is very exothermic and temperature sensitive. Thus, significant attention must be provided to the reactor design.

Numerous challenges are encountered in the design and construction of a commercial scale chemical reactor. A slurry bubble column reactor (“SBCR”) has been shown to display numerous advantages over a fixed bed reactor. For example the SBCR has a very high heat transfer capacity and allows for very stable operations even with a highly exothermic reaction. The SBCR also makes it possible to remove a small portion of catalyst for regeneration and/or replacement without shutting the reactor down. This makes it possible to achieve a higher percentage of run time than a fixed bed reactor. A SBCR utilizes relatively small catalyst particles, which is highly effective with a diffusion-controlled reaction. The practical size range for catalyst particles in a SBCR is much smaller than what can be considered for a fixed bed reactor.

A SBCR, however, has several limitations. For example, a maximum practical vessel diameter is set by construction and transport limitations. If the need arises to increase the reactor volume, the volume increase is typically accomplished by making the reactor taller. A practical height limit will quickly be reached. Adding incrementally to the height of the reactor adds significantly to the cost of the reactor, because incremental height adds to foundation and structural costs and makes operation more difficult.

Another limitation of the conventional SBCR is its use on a barge or ship where the vertical height of a reactor and weight of the slurry add significantly to the cost of installation and greatly complicate its operation.

In general, it would be desirable to provide a means for conducting various reactions using a low cost reactor that is easy to build and maintain. In particular, a reactor that is not excessively tall in height, and provides a large throughput, would be desirable. A reactor which does not need to be shut down in order to replace or replenish catalyst would be very desirable. The industry is in need of a relatively inexpensive and efficient reaction system and process that is capable of removing intermediate reaction products or by-products, i.e., water, during the reaction to boost efficiency of the conversion of syngas into hydrocarbon products. This invention is directed to provide apparatus and processes which will address the above concerns.

### SUMMARY OF THE INVENTION

Therefore, a need has arisen to provide a method and apparatus that addresses the shortcomings of prior art methods and apparatus. It is one object of this invention to provide a low profile moving bed reactor employing one or more parallel reaction zones where reactants flow vertically up through a slurry catalyst system in a horizontal vessel which has significant advantages for more efficient transportation, set-up, installation operation and lower manufacturing costs.

It is another object of the invention to provide a low incremental capital cost for the deployment of equipment needed to process syngas into hydrocarbons.

It is yet another object of the invention to provide a low cost method of processing syngas using multiple stages in a reactor design that is oriented in the horizontal direction.

It is a further object of the invention to provide a reactor design and system that can be used with multiple passes in a multi-stage reaction process in one vessel.

In one aspect of the invention, a slurry configuration may be processed in a design that is not as severely throughput limited, that is, in which a larger volume of gas may be processed in a single reaction vessel.

In one aspect of the invention, it is possible to use heat transfer bundles which project from either or both ends of the reactor vessel into the reaction zone. Such heat transfer bundles may be used to remove heat from the reaction system.

In another aspect of the invention, it is possible to use external heat transfer bundle to remove heat from the circulating slurry stream.

In another aspect of the invention, it is an object to provide for a reactor that is easier to maintain and provides greater safety for maintenance personnel engaged in routine maintenance.

In another aspect of the invention, a reactor design is provided which is capable of processing syngas into hydrocarbons by removing water from the system during the reaction process, and then re-injecting the process stream back into the reactor for further conversion, increasing efficiency of conversion.

It is one object of the invention to provide a reactor design that is oriented horizontally so as to be more stable if floated upon a ship or barge, such as when the reactor is employed in off-shore operations.

It is yet another object of the invention to provide a reactor design that is capable of processing syngas at an efficient rate, and does not require a shut-down of the reactor to replenish or replace the catalyst.

A moving bed or slurry bubble column reactor having a longitudinal axis substantially perpendicular to the gravity field (i.e. horizontal) is provided. A generally cylindrical body having a first end and a second end is used, in which the cylindrical body has an inlet for receiving a gaseous feedstock and a fluid outlet for removing unreacted gases and light reaction products. Liquid reaction products are removed from the circulating slurry. Furthermore, a first reaction zone is adapted for upward movement of gaseous feedstock to form a first process fluid. Then, a first separation means is afforded for removing water from the first process fluid to form a dewatered first process fluid. A second optional reaction zone is adapted for upward movement of a dewatered first process fluid to form a second process fluid.

In another embodiment of the invention, a moving bed reaction system having multiple reaction zones arranged in parallel is provided which includes a reactor body having a first end and a second end. The reactor body has an inlet for receiving a gaseous feedstock and a fluid outlet for removing a processed fluid. Furthermore, a first reaction zone is adapted for upward movement of gaseous feedstock. The first reaction zone comprises a catalyst and a liquid slurry. The gaseous feedstock is exposed to catalyst in the liquid slurry. Further, the gaseous feedstock forms a first process fluid. A second reaction zone substantially parallel to the first reaction zone is also provided. The second reaction zone is adapted for upward movement of the first process fluid. The second reaction zone comprises catalyst and liquid slurry, the first process fluid being converted to form a second process fluid in the course of the process. A first baffle is disposed between the first reaction zone and the second reaction zone, whereby the first baffle is discontinuous along its length. The first baffle is adapted to equalize pressure between the first reaction zone and the second reaction zone. Furthermore, compression energy may be added to the gaseous feedstock exiting the first reaction zone to equalize the pressure between the zones. Subsequent zones may be operated in a like manner.

### BRIEF DESCRIPTION OF THE DRAWINGS

A full and enabling disclosure of this invention, including the best mode shown to one of ordinary skill in the art, is set forth in this specification. The following Figures illustrate the invention:

FIG. 1 shows a schematic diagram of the exterior of a low profile moving bed reactor of the invention;

FIG. 2 provides a longitudinal cross-sectional view of one embodiment of the reactor of the invention as shown in FIG. 1;

FIG. 3 provides a radial cross section of one embodiment of the reactor that may be employed, in the invention;

FIG. 4A shows a radial cross section of the reactor of FIG. 2 taken along line 4A-4A of FIG. 2;

FIG. 4B shows an alternate embodiment of the reactor design (radial cross-section) of the invention in which one or more openings in the baffle are circular rather than longitudinal; and

FIG. 4C shows yet another alternate embodiment of the reactor design (radial cross-section) in which the baffle has a plurality of openings to facilitate pressure equalization between a first reaction zone and a second zone.

### DETAILED DESCRIPTION OF THE INVENTION

Reference now will be made to the embodiments of the invention, one or more examples of which are set forth below. Each example is provided by way of explanation of the invention, not as a limitation of the invention. In fact, it will be apparent to those skilled in the art that various modifications and variations can be made in this invention without departing from the scope or spirit of the invention. For instance, features illustrated or described as part of one embodiment can be used on another embodiment to yield a still further embodiment. Thus, it is intended that the present invention cover such modifications and variations as come within the scope of the appended claims and their equivalents. Other objects, features and aspects of the present invention are disclosed in or are obvious from the following detailed description. It is to be understood by one of ordinary skill in the art that the present discussion is a description of exemplary embodiments only, and is not intended as limiting the broader aspects of the present invention, which broader aspects are embodied in the exemplary constructions.

A SBCR is limited to a maximum throughput set by the cross sectional area of the reactor. Some practical limit is reached due to fabrication limits, and thus the reactor capacity can only be further increased by adding additional parallel units, as shown by the invention.

A SBCR is limited to a single stage within one shell. Multiple stages can be accomplished by connecting several shells in series. Some reactions that produce a by-product benefit from the ability to remove by-products one or more times along the reaction path. For example, in a Fischer-Tropsch reaction, water is generally produced as a by-product and it may be desirable to remove water before allowing the reaction to go to completion. This can be done in a conventional SBCR only by establishing a recycle loop. This results in reducing the fresh feed throughput of the reactor due to the amount of recycle gas that must be included to operate within a practical limit of conversion.

In the case of a nitrogen diluted syngas stream, the partially converted gas cannot be recycled back due to the build up of nitrogen that would occur. Therefore multi-stage operation would be necessary, which has the limitations already described in a conventional SBCR.

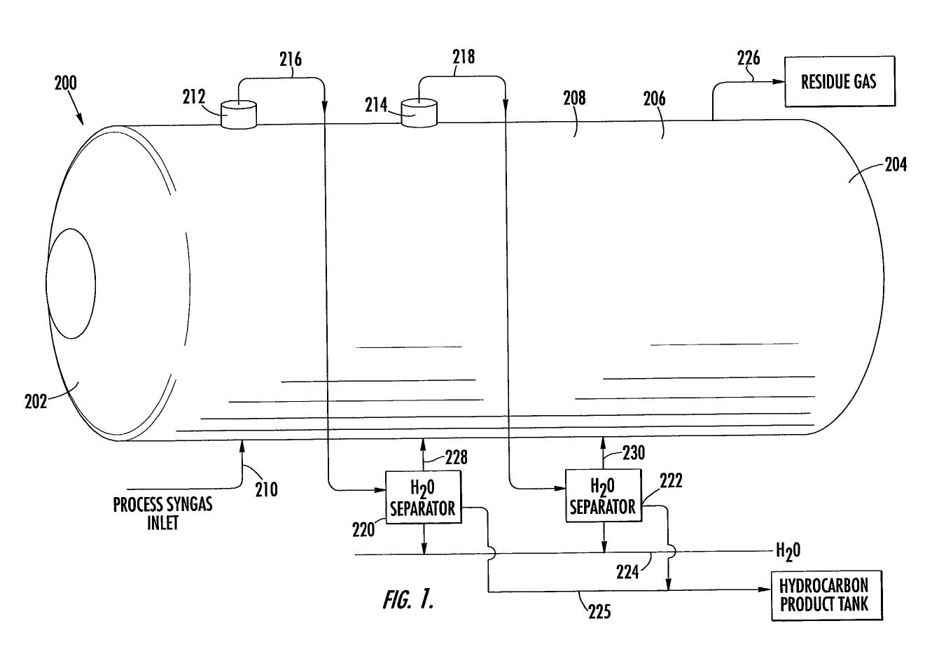

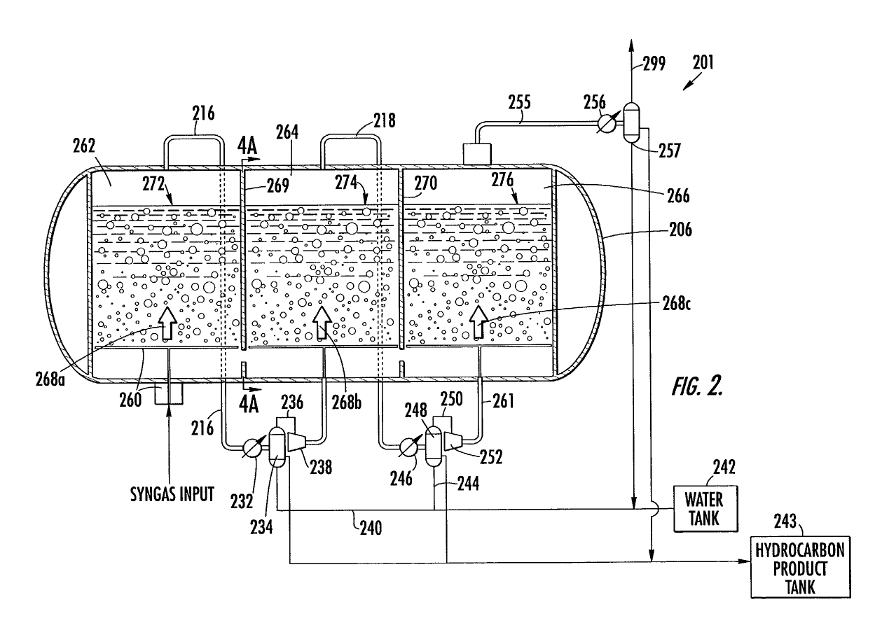

FIG. 1 shows a schematic diagram of the exterior of a low profile SBCR moving bed reactor of the invention. A low profile moving bed reactor 200 is shown. A first end 202 on the left side of FIG. 2 and a second end 204 on the right side of FIG. 2 form the end caps of the cylindrical housing 206. Although other geometric arrangements of the housing could be provided, a cylindrical body 208 has been shown to work well in one application of the invention.

By way of example, the reactor may be described for use as a Fischer-Tropsch reactor. In that application, a feedstock process inlet 210 provides the incoming stream of synthesis gas “syngas” into a fluid slurry or process stream inside the reactor. The gas bubbles up through the process slurry as will be further shown in connection with FIG. 2. The gas reacts with catalysts during its ascension through the fluid slurry. Catalyst is provided in a powder form within the fluid slurry, and thus the syngas is well mixed with the slurry within the reactor vessel itself. The process fluid within the reactor contains varying levels of feedstock gas, desired product, unreacted synthesis gas, water and inert compounds depending upon the location of the process fluid within the reactor 200. Near the inlet 210 the process fluid will consist almost entirely of feedstock, while near residue gas outlet 226, the process fluid at that point is depleted of reactants. Thus, as the process fluid flows through the reactor 200, one or more additional substances or reactants may be added or removed at selected locations in accordance with teachings of the present invention.

The reactor 200 may have a plurality of baffles, or separators, such as first baffle 269 and second baffle 270 shown in FIG. 2.

A slurry recycle loop (not shown in FIG. 1) may be employed as an optional feature of the invention. This loop would remove product and catalyst, filter or separate a portion of it, and return concentrated slurry to the reactor. Such a loop is also useful for maintaining catalyst dispersion in the reactor and may be used with a heat exchanger to reuse heat via an external heat exchanger.

Turning back to FIG. 1, the process fluid moves along the first process fluid conduit 216 to separator 220 where water and light hydrocarbon product may be taken from the process fluid and provided to water line 224 and product line 225 for removal. The remaining dewatered first process fluid is provided along first return line 228 back into the reactor 200. Then, the dewatered first process fluid is bubbled up through the slurry and is provided through a different reaction zone to a point at second inlet 214 where it follows second process fluid conduit 218 to separator 222. Then, water and product may be separated out and provided to water line 224 and product line 225 and the dewatered second process fluid proceeds back into the reactor along second return line 230 where it again is bubbled up through the fluid slurry in the reactor, eventually escaping by way of residue gas outlet 226. The removed process water may contain some contaminants and is delivered to a water treatment system.

There may be one, two, three, or more reaction zones within the reactor, and there is no practical limit on the number of times the process fluid may be removed from the reactor, water and products separated from the fluid, and then returned to the reactor. In fact, it will sometimes be possible and advantageous to remove fluid from the reactor, remove water and other products from the fluid. In other embodiments, more than one reactor can be used in which fluid is taken from a first reactor, products or by products removed or processed, and then placed into a second reactor for further processing. Likewise, heat generated during the course of the reaction may be transferred by way of one or more heat generators to do work at another point in the system, or to simply provide heat energy to be stored or used in another application.

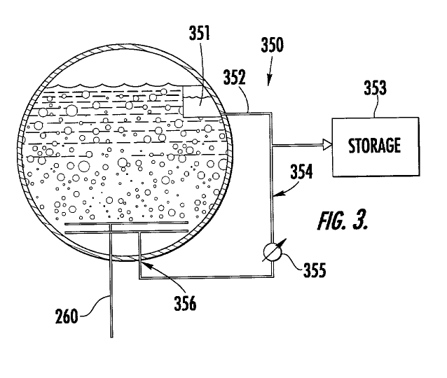

In FIG. 2, syngas is provided as an input to the low profile moving bed reactor 200 at inlet distributor 260. The syngas bubbles up into the first reaction zone 262 along direction arrow 268a as shown in FIG. 3. The syngas reacts in the fluid slurry until it reaches the first fluid level 272 near the top of first reaction zone 262.

In FIG. 2, unreacted syngas and conversion products from the first zone is cycled outside the reactor along to conduit 216 to a first heat exchanger 232. In this embodiment, a heat exchanger is used to cool the syngas condensing some of the reaction products and water. Then, the first process stream is provided to first separator 234, which separates some of the reaction water and products and provides it along conduit 240 from the dewatered first process fluid which is provided along conduit 236 into a first compressor 238. The first compressor 238 pumps the partially converted syngas stream back into the lower portion of the second reaction zone 264, where the dewatered first process stream is again reacted in a fluid slurry until it reaches the second fluid level 274 near the top of the second reaction zone 264. The first reaction zone 262 and the second reaction zone 264 are separated by first baffle 269 which may contain openings or discontinuities in its surface, such as that shown in FIG. 2. Such discontinuities or openings allow slurry to communicate between the zones.

Unreacted syngas from the second zone enters conduit 218 where it is provided outside of the reactor 200 into a second heat exchanger 246, seen near the bottom of FIG. 2. Then, the second process stream is provided to second separator 248, where water and hydrocarbon product is removed and diverted to tanks 242 and 243 respectively. The process stream is provided along conduit 250 and into second compressor 252. The dewatered second process stream is compressed and pumped from second compressor 252 into conduit 261 which returns the fluid back into a third reaction zone 266. The remaining reactants again react with the slurry catalyst in zone 266. Any remaining unreacted syngas and vapor phase water and hydrocarbon product exits the reactor into conduit 255 where it is provided to a third heat exchanger 256, and a third separator 257 where the water and hydrocarbon product is removed and diverted to tanks 242 and 243 respectively. Any remaining non-condensible residue exists in the reactor system in conduit 299.

A second baffle 270 separates the second reaction zone 264 from the third reaction zone 266. There is no limit to the number of reaction zones that can be provided in the reactor of the invention, and the embodiment shown in FIG. 2 with three zones is but one example of the arrangement that can be employed in the invention. In fact, the number of reactor zones could be as few as one. There is no limit to the number of separators, heat exchangers, or compressors that may be used in the invention, and there may be one or more of each employed in the invention. Futhermore, separated water line 244 and conduit 240 provide water into storage 242 or to a water treatment system.

The slurry system may be mixed solely by the energy provided by the inlet gas or optionally the slurry is allowed to collect and degas in the upper portion of the reactor trough in FIG. 3. FIG. 3 shows a cross sectional view of one embodiment of the reactor of the invention in which a recycled loop 350 is employed. Material is pulled into the trough 351, flows into the recycle line 352, and long chain hydrocarbons or waxes, including also the catalyst material, is pulled off into storage 353. Otherwise, material passes along line 354, through the cooler 355 and the slurry reenters the reaction vessel at entry point 356 where the gaseous and light hydrocarbon material is bubbled up through the slurry once more. The degassed slurry has increased density and recycles to the bottom of the reactor. In this configuration the slurry system remains well mixed. Hydrocarbon product thus can be removed from the degassed slurry stream. In either case a relatively large volume of slurry and concentrated slurry is returned to the reactor. This slurry removal product separation process and apparatus may be provided to any or all zones of the reactor.

In general, the reaction of the catalyst with the syngas is a secondary liquid phase reaction occurring in the fluid as the gas bubbles up through the fluid in each respective reaction zone. An appropriate catalyst may be disposed within the process fluid and in general in most cases will be dispersed within the fluid. Furthermore, it is possible to remove the catalyst and regenerate or replace the catalyst outside of the reactor, in a continuous process that does not require shut down of the reactor in order to regenerate or replace the catalyst used in the reaction.

In most cases, the catalyst comprises one of the cobalt catalysts that is known to be capable of reacting syngas into hydrocarbon products such as paraffins, olefins, and oxygenates. However, other catalysts can be used, and the invention is not limited to cobalt catalysts.

Above certain conversion levels such as 50-60%, the excess water in the reaction system begins to reduce the efficiency of the reaction. Thus, one advantage of the invention is that it provides a method to remove water from the reaction, as it is reacting and before it has completed its reaction. Using nitrogen diluted gas means that the recycling is limited because nitrogen would build up excessively in that case. Therefore, the invention has the advantage of removing water from the reaction as the reaction proceeds in a multi-stage series process but within a single reactor. In this way, it is possible to achieve overall conversion into product in the 85-95% range. This is efficiently achieved by using multiple series stages which are aligned in parallel along the longitudinal horizontal axis of a single reactor, instead of in a tall reactor vertically oriented.

The low profile reactor of this invention provides advantages that include easier transportation of the reactor, and reduced installation costs. The reactor can be made longer with a lower incremental cost per pound of product produced, whereas a taller reactor would require more expense per pound of product produced because of the necessity to have a more robust foundation and a more expensive tower configuration.

The operation of the reactor in this invention may be one stage, two stages, three stages, four stages, or more depending upon the configuration used in the invention. Furthermore, the slurry configuration is not gas throughput limited, and the geometry of the reaction vessel for a given zone may facilitate a larger cross sectional area and a relatively larger capacity for the reactor to expand by making the zone longer, since the cross sectional area of the reactor zone is defined by multiplying the width of the cross section of the reactor (at the point where the gas distributor is installed above the bottom of the reactor) by the length of a reactor zone.

Heat transfer bundles (not shown) optionally may be provided on the first end 202 or the second end 204 of the reactor or may extend the entire length of the vessel going through the baffles that separate zones. Such heat transfer bundles, which are not shown in the Figures, may proceed from the end of the reactor into the reactor and serve to take heat out of the reaction system as it is proceeding, serving to cool and maintain the appropriate temperature of the reactants in solution. Furthermore, heat transfer may also be provided in the sides of the reaction vessel using other known means to remove or transfer heat from a reaction vessel with liquid circulation shown in FIG. 3.

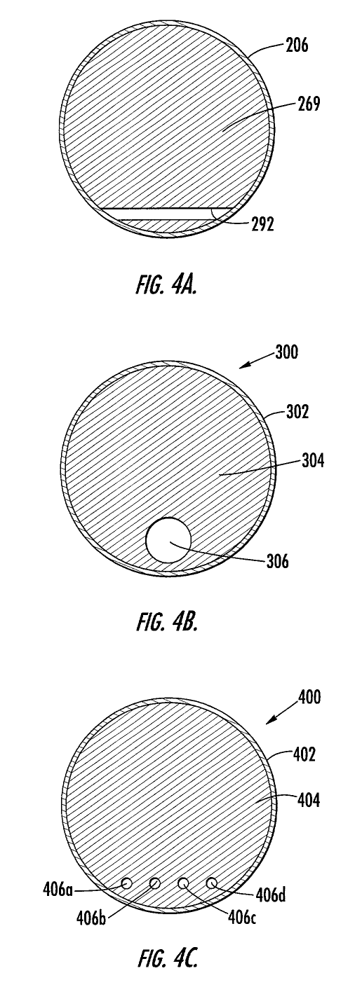

Turning now to FIG. 4A, a radial cross section of the reactor taken along lines 4A-4A in FIG. 3 is shown in which a housing 206 contains a first baffle 269 having an opening 292 near the lower portion of the first baffle 269. Process fluid (slurry) may pass through the first baffle 269 along the opening 292 in the first baffle 269.

In another configuration of the invention, baffles may be provided as shown in FIG. 4B which shows an alternate reactor design 300 having a baffle 304 which is within housing 302 of the reactor, and contains an opening 306 in the baffle.

In yet another configuration of the invention shown in FIG. 4C, alternate reactor design 400 is provided with a housing 402 containing a baffle 404. Baffle openings 406a, 406b, 406c, and 406d, and perhaps others, are provided along the lower margin of the baffle 404 to provide fluid communication from one reaction zone into another reaction zone.

It is understood by one of ordinary skill in the art that the present discussion is a description of exemplary embodiments only, and is not intended as limiting the broader aspects of the present invention, which broader aspects are embodied in the exemplary constructions. The invention is shown by example in the appended claims.

What is claimed:

1. A slurry bubble column reactor, comprising:
a generally cylindrical body having a first end and a second end, the cylindrical body having a longitudinal axis substantially perpendicular to the gravity field (i.e. horizontal) and having an inlet for receiving a gaseous feedstock and a fluid outlet for removing a process fluid;
a first reaction zone adapted for upward movement of gaseous feedstock in a direction that is substantially perpendicular to the longitudinal axis to form a first process fluid;

2. The reactor of claim 1 further comprising:
a first separation means for removing water from the first process fluid to form a dewatered first process fluid.

3. The reactor of claim 1 additionally comprising:
a second reaction zone adapted for upward movement of a dewatered first process fluid to form a second process fluid.

4. The reactor of claim 3 additionally comprising at least one baffle adapted to segment the reactor into more than one zone.

5. The reactor of claim 3 in which a first baffle separates the first reaction zone from the second reaction zone.

6. The reactor of claim 3, further comprising:
a means for removing products of the reaction between the first and second zones of the reactor.

7. The reactor of claim 1 in which the gaseous feedstock is syngas.

8. The reactor of claim 1 in which the reactor further comprises a catalyst for converting syngas to hydrocarbons.

9. The reactor of claim 3 further comprising at least one heat exchanger.

10. The reactor of claim 3 whereby the catalyst may be replenished or regenerated in a continuous cycle.

11. The reactor of claim 3 in which the first baffle is discontinuous along its length, thereby facilitating pressure equalization between the first reaction zone and the second reaction zone.

12. The reactor of claim 9 in which the fluid level in the first reaction zone and the fluid level in the second reaction zone remain substantially the same.

13. A moving bed reaction system having multiple reaction zones arranged in parallel, comprising:
a reactor body having a first end and a second end, the reactor body having a longitudinal axis oriented horizontally, and an inlet for receiving a gaseous feedstock and a fluid outlet for removing a processed fluid;
a first reaction zone adapted for upward movement of gaseous feedstock, the first reaction zone comprising a catalyst and a liquid slurry, the gaseous feedstock being exposed to catalyst in the liquid slurry, the gaseous feedstock thereby forming a first process fluid;
a second reaction zone substantially parallel to the first reaction zone, the second reaction zone adapted for upward movement of the first process fluid, the second reaction zone comprising catalyst and liquid slurry, the first process fluid being converted to form a second process fluid; and
a first baffle disposed between the first reaction zone and the second reaction zone, whereby the first baffle is discontinuous along its length such that the first baffle is adapted to equalize pressure between the first reaction zone and the second reaction zone.

14. The system of claim 13 in which reaction products and/or by products are removed from the first process fluid prior to entry of the first process fluid into the second reaction zone.

15. The system of claim 13 in which the gaseous feedstock comprises syngas.

16. The system of claim 13 additionally comprising:
a third reaction zone adapted for upward movement of the second process fluid, the third reaction zone comprising catalyst and liquid slurry, the second process fluid being converted in the third reaction zone to form a third process fluid; and
a second baffle disposed between the second reaction zone and the third reaction zone, whereby the second baffle is discontinuous along its length such that the second baffle is adapted to equalize pressure between the second reaction zone and the third reaction zone.

17. The system of claim 13 in which the first baffle comprises at least one opening in its surface such that the liquid slurry may pass freely from the first reaction zone to the second reaction zone through the opening in the first baffle.

18. A process for synthesizing hydrocarbons from syngas in a multi-stage operation comprising the steps of:
(a) providing a reaction vessel having first and second reaction zones arranged horizontally;
(b) providing syngas feedstock into the lower portion of the first reaction zone;
(c) providing a catalyst and liquid slurry in the first and second reaction zones;
(d) reacting the syngas feedstock with the catalyst in the first reaction zone while bubbling the syngas feedstock upwards, thereby forming a first process fluid;
(e) diverting said first process fluid from the first reaction zone to a second reaction zone; and
(f) reacting said first process fluid in the second reaction zone with catalyst while passing the first process fluid upwards, thereby forming a second process fluid containing hydrocarbon products.

19. The process of claim 18 further comprising the step of removing reaction products from the first process fluid after it is diverted from the first reaction zone, and before it is provided into the second reaction zone.

20. The process of claim 18 in which the reaction comprises a first baffle between the first reaction zone and the second reaction zone.

21. The process of claim 20 in which mixing of the slurry between the first reaction zone and the second reaction zone is facilitated by openings in the first baffle.

22. The process of claim 18 in which the first process fluid moves from the first zone to the second zone, thereby equalizing pressure between the first and second zones.

23. The process of claim 18 further comprising a third reaction zone.
